(19) United States
(12) Patent Application Publication
(10) Pub. No.: US 2009/0247570 A1
(43) Pub. Date: Oct. 1, 2009

(54) PHARMACEUTICALS

(75) Inventor: Howard Bernard Mayer, New York, NY (US)

Correspondence Address: PFIZER INC;Mary J Hosley, 150 EAST 42ND STREET, MS: 150/02/E112, NEW YORK, NY, 10017-5612, US

(73) Assignee: Pfizer Inc

(21) Appl. No.: 12/304,400

(22) PCT Filed: May 30, 2007
(86) PCT No.: PCT/IB07/01508

§ 371 Date: Dec. 11, 2008

Related U.S. Application Data
(60) Provisional application No. 60/804,480, filed on Jun. 12, 2006.
(60) Provisional application No. 60/821,974, filed on Aug. 10, 2006.
(60) Provisional application No. 60/888,387, filed on Feb. 6, 2007.

(51) Int. Cl. A61K 31/437; A61K 31/439

(52) U.S. Cl. 514/303; 514/299

(57) ABSTRACT

The present invention relates to the use of a CCR5 antagonist in an HIV infected patient to enhance their immune reconstitution and so treat to HIV related opportunistic conditions resulting from the immunocompromised state of the HIV patient. The invention also allows treatment with a CCR5 antagonist of patients having a CXCR4 using viral population since such patients will also benefit from an increase in their CD4 and/or CD8 cell count.

This invention relates to the use of a CCR5 modulator, in particular an antagonist, to enhance the immune reconstitution of a patient infected with HIV such as HIV-1, and genetically related retroviral infections, and the resulting acquired immune deficiency syndrome, AIDS. The invention further includes the use of combinations containing a CCR5 antagonist for the treatment of HIV and AIDS patients.

The name “chemokine”, is a contraction of “chemotactic cytokines”. The chemokines comprise a large family of proteins which have in common important structural features and which have the ability to attract leukocytes. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory and infectious diseases, agents which are active in modulating, preferably antagonizing, the activity of chemokines and their receptors, are useful in the therapeutic treatment of such inflammatory and infectious diseases.

The chemokine receptor CCR5 is of particular importance in the context of treating inflammatory and infectious diseases. CCR5 is a receptor for chemokines, especially for the macrophage inflammatory proteins (MIP) designated MIP-1α and MIP-1β, and for a protein which is regulated upon activation and is normal T-cell expressed and secreted (RANTES).

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus III (HTLV-III), now more commonly referred to as the human immunodeficiency virus or HIV.

HIV is a member of the class of viruses known as retroviruses. The retroviral genome is composed of RNA which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for human T-4 lymphocyte (CD4) and CD8 cells which plays a vital role in the body's immune system. HIV infection of the white blood cells depletes this white cell population. Eventually, the immune system is rendered inoperative and ineffective against various HIV-related opportunistic conditions, such as pneumocystitis carini, Kaposi's sarcoma and cancer of the lymph system.

The CD4 cell has both CCR5 and CXCR4 co-receptors on its surface, which it is thought HIV uses to gain entry to the cells. However different populations of the virus exist and can be classified according to the co-receptor (CCR5 or CXCR4) which they would normally use for cell entry. Hereinafter viral populations containing substantially CCR5 virus are classified as CCR5 tropic. Viral populations containing substantially CXCR4 virus are classified as CXCR4 tropic, viral populations with both CCR5 and CXCR4 virus are classified as mixed tropic, while a dual tropic virus can enter the CD4 cell via either the CCR5 or CXCR4 co-receptor.

Hereinafter a CXCR4 using viral population is classified as that containing some CXCR4 virus, preferably more than 2% CXCR4 virus, more preferably more than 5% CXCR4 virus, most preferably more than 10% CXCR4 virus.

HIV patients who have not received any previous HIV drug therapy are classified as treatment naive patients and are generally infected predominantly with the CCR5 virus, while those patients who have received some HIV drug therapy are classified as treatment experienced patients. Over time, treatment experienced patients tend to build up a resistance to many HIV drugs and, perhaps coincidently, there appears to be an increasing emergence of CXCR4 virus. Eventually a treatment experienced patient will progress to AIDS when the immune system can no longer be maintained. Koot et al (Ann. Intern. Med. 1993; 118:681-688) reported on a two and a half year study following almost 200 treatment experienced HIV patients receiving nucleoside/nucleotide reverse transcriptase inhibitors (NRTI). Two classes of patient were identified: those infected with syncytium inducing (SI) virus (a form of CXCR4) and those without SI virus (i.e. the CCR5 tropic patient population). The authors reported that the probability of progression to AIDS in the patients with SI virus was 70.8% whereas the probability for progression to AIDS in patients without the SI virus was 15.8%. Additionally, the appearance of SI variants was said to be prognostic for the rapid decline of CD4 cell count.

Therefore it has been thought that the CXCR4 virus is the more damaging to CD4 cells and could accelerate the onset of AIDS, such as by increasing the decline of CD4 cells. Accordingly, there was a concern that a drug which only inhibited CD4 cell entry of CCR5 tropic virus may do harm in patients infected with a CXCR4 using viral population. That is, the selective suppression of the CCR5 tropic virus may, by allowing additional cellular targets for the CXCR4 tropic virus, accelerate the infection of CD4 cells thereby allowing an increase in viral load and a decline in CD4 count. This, in turn, could accelerate the onset of AIDS and HIV related opportunistic conditions.

An assay has therefore been developed to determine the tropism of the viral population that HIV patients are infected with, and accordingly provide appropriate treatment.

Maraviroc, chemical name, (N-{(1S)-3-[3-isopropyl-5-methyl-4H-1,2,4-triazole-4-yl]-exo-8-azabicyclo[3.2.1]oct-8-yl}-1-phenylpropyl)-4,4-difluorocyclohexanecarboxamide), which is disclosed in WO 01/90106 (incorporated herein by reference), is a chemokine receptor antagonist which inhibits entry of HIV through the CCR5 co-receptor (i.e. maraviroc is a CCR5 antagonist) and is indicated for treatment of patients infected with CCR5 tropic HIV.

The Phenosense™ assay, otherwise known as the Trofile™ assay, (Monogram Biosciences, California, USA) can be used to determine if an HIV patient is CCR5 tropic, and if so, maraviroc can then be administered. Maraviroc is not indicated for non CCR5 tropic (i.e. CXCR4 tropic, dual/mixed tropic) and maraviroc or any other CCR5 antagonist would not be expected to be of therapeutic benefit to these HIV patients.

We have now found that use of a CCR5 antagonist in patients infected with a CXCR4 using viral population induces a clinically meaningful increase of CD4, or CD8, or both CD4 and CD8, cell count.

This increase in cell count indicates that use of CCR5 antagonist can enhance the immune reconstitution of an HIV patient regardless of their viral tropism. By enhancing the immune reconstitution we mean that the patient's immune function can be recovered to levels for the treatment of HIV related opportunistic conditions.

Thus a CCR5 antagonist could beneficially treat all HIV patients and could be administered without having to first determine their viral tropism.

HIV related opportunistic conditions are those conditions to which HIV infected patients are more susceptible as a result of their immunocompromised state.

By treatment we mean to include that the risk of contracting an HIV related opportunistic condition is reduced and the patient's ability to fight existing HIV related opportunistic conditions is improved. Thus treatment includes both treatment and prophylaxis of HIV related opportunistic conditions.

The increased CD4, or CD8, or both CD4 and CD8, cell count may also delay the onset of AIDS in the HIV patient.

Accordingly, in a first aspect of the invention, there is provided the use of a CCR5 antagonist in the preparation of a medicament to enhance the immune reconstitution of a patient infected with HIV.

In accordance with the first aspect of the invention, enhanced immune reconstitution is measured by recovery of CD4 cell count. The size of increase in the CD4 cell count will depend on the individual patient, with the patient increasingly benefiting from immune reconstitution the higher the level of CD4 cell count that can be achieved. For example, an HIV patient with a very low CD4 cell count of say 10 cells/μL would still benefit clinically from an increase to about 50 cells/μL.

In a second aspect of the invention, there is provided the use of a CCR5 antagonist in the preparation of a medicament to increase the CD4, or CD8, or both CD4 and CD8 cell counts, in a patient infected with HIV.

In a third aspect of the invention, there is provided the use of a CCR5 antagonist in the preparation of a medicament for use as an immunepotentiator in a patient infected with HIV, such as a CXCR4 using viral population.

In a fourth aspect of the invention, there is provided the use of a CCR5 antagonist in the preparation of a medicament for the treatment of an HIV related opportunistic condition.

The following embodiments and all combinations thereof relate to and independently further define the first, second and third aspects of the invention, unless stated otherwise.

In one embodiment of the first to third aspects of the invention, the medicament is provided to the HIV patient for the treatment of HIV related opportunistic conditions.

In a further embodiment, the CD4 count of the patient before administration of the CCR5 antagonist (i.e. the baseline CD4 count) is 400 cells/μL. In yet a further embodiment, the baseline CD4 count is 200 cells/μL. In a yet further embodiment, the baseline CD4 count is 50 cells/μL.

In a yet further embodiment, the CD4 count of the patient after treatment with a CCR5 antagonist is increased to more than 50 cells/μL. In a yet further embodiment, the CD4 count of the patient after treatment with a CCR5 antagonist is increased to more than 100 cells/μL. In a yet further embodiment, the CD4 count of the patient after treatment with a CCR5 antagonist is increased to more than 200 cells/μL. In a yet further embodiment, the CD4 count of the patient after treatment with a CCR5 antagonist is increased to more than 350 cells/μL. In a yet further embodiment, the CD4 count of the patient after treatment with a CCR5 antagonist is increased to more than 600 cells/μL.

In a yet further embodiment, the CD4 cell count after treatment with the CCR5 antagonist is increased by more than 60% over the baseline cell count. In a yet further embodiment, the increase in CD4 cell count is 100% over baseline cell count. In a yet further embodiment, the increase in CD4 cell count is 200% over baseline cell count.

The increase in cell count manifests itself in the circulating blood of the HIV patient, but there may also be an increased cell count in other parts of the body (e.g. the lymph gland).

In a yet further embodiment, the patient is treatment experienced (but not receiving a CCR5 antagonist).

In a yet further embodiment of the invention, the patient is infected with a CCR5 tropic viral population, is treatment experienced (but not receiving a CCR5 antagonist) and has a low viral load (such as where the patient has already responded viralogically and has a viral load which is largely under control). In accordance with the invention, a CCR5 antagonist can now be given to the patient as a further agent (add on therapy) to increase CD4 cell count and thus enhance their immune reconstitution.

In a yet further embodiment, the HIV patient has an HIV viral load of more than 5000 copies\mL. In a yet further embodiment, the HIV patient has an HIV viral load of more than 1000 copies\mL.

In a yet further embodiment, the HIV patient has an HIV viral load of less than 5000 copies\mL. In a yet further embodiment, the HIV patient has an HIV viral load of less than 400 copies\mL. In a yet further embodiment, the HIV patient has an HIV viral load of less than 200 copies\mL. In a yet further embodiment, the HIV patient has an HIV viral load of less than 50 copies\mL.

In a yet further embodiment of the invention, the HIV patient is infected with a CXCR4 using viral population. These patients will normally be on existing HIV drug therapy and would not receive a CCR5 antagonist. However in accordance with the invention, the CCR5 antagonist may be given as a further agent (add on therapy) to their existing HIV drug regime.

In a yet further embodiment of the invention, the viral population of the HIV patient contains more than 2% CXCR4 virus. In a yet further embodiment of the invention, the viral population of the HIV patient contains more than 5% CXCR4 virus. In a yet further embodiment of the invention, the viral population of the HIV patient contains more than 10% CXCR4 virus. In a yet further embodiment of the invention, the viral population of the HIV patient contains more than 15% CXCR4 virus. In a yet further embodiment of the invention, the viral population of the HIV patient contains more than 20% CXCR4 virus. In a yet further embodiment of the invention, the viral population of the HIV patient contains more than 25% CXCR4 virus. In a yet further embodiment of the invention, the viral population of the HIV patient contains more than 30% CXCR4 virus. In a yet further embodiment of the invention, the viral population of the HIV patient contains more than 35% CXCR4 virus. In a yet further embodiment of the invention, the viral population of the HIV patient contains more than 40% CXCR4 virus. In a yet further embodiment of the invention, the viral population of the HIV patient contains more than 45% CXCR4 virus. In a yet further embodiment of the invention, the viral population of the HIV patient contains more than 50% CXCR4 virus.

In a yet further embodiment of the invention, the HIV patient is infected with a CCR5 tropic viral population.

HIV related opportunistic conditions include opportunistic infections and malignancies.

Examples of HIV related opportunistic conditions include pneumocystitis carnii, toxoplasmosis, isoporiasis, cryptosporidiosis, cadidiasis, cryptococcosis, histoplasmosis, coccidioidomycosis, mycobacterium tuberculosis, non tuberculosis mycobacterium infections, salmonella, cytomegalovirus, herpes simplex virus, recurrent or persistent upper respiratory infection, sinuisitis, otitis media, bacterial meningitis, pneumonia, sepsis, oropharyngis candidaiasis, diarrhea, hepatitis, herpes zoster, leiomyosarcoma, lymphoid interstiticial pneumonia, nocardiosis, disseminated varicella, and toxoplasmosis of the brain, progressive multifocal leukoencephalopathy, Kaposi's sarcoma, lymphoma, cervical cancer, HIV dementia and HIV wasting syndrome.

More particularly, examples of HIV related opportunistic infections include pneumocystitis carnii, toxoplasmosis, isoporiasis, cryptosporidiosis, cadidiasis, cryptococcosis, histoplasmosis, coccidioidomycosis, mycobacterium tuberculosis, non tuberculosis mycobacterium infections, salmonella, cytomegalovirus, herpes simplex virus, progressive multifocal leukoencephalopathy, recurrent or persistent upper respiratory infection, sinuisitis, otitis media, bacterial meningitis, pneumonia, sepsis, ordpharyngis candidaiasis, diarrhea, hepatitis, herpes zoster, leiomyosarcoma, lymphoid interstiticial pneumonia, nocardiosis, disseminated varicella, and toxoplasmosis of the brain.

Examples of malignancies are progressive Kaposi's sarcoma, lymphoma and cervical cancer.

In a yet further embodiment, the CCR5 antagonist has an IC50 for CCR5 binding of less than 1 μM (as determined by the MIP-1β assay of Combadiere et al, J. Leukoc. Biol., 60, 147-152 (1996)).

In a yet further embodiment of the invention, the CCR5 antagonists is selected from maraviroc, NCB-9471, PRO-140, CCR5mAb004, TAK-779 (WO 99/32468), ZM-688523, 4-chloro-6-fluoro sulphonamide, TAK-220 (WO 01/25200), TAK-652 which is disclosed in WO03014105 and has the chemical name 8-[4-(2-butoxyethoxy)phenyl]-1-isobutyl-N-[4-[[(1-propyl-1H-imadazol- 5-yl)methyl]sulphinyl]phenyl]-1,2,3,4-tetrahydro-1-benzacocine-5-carboxamide, SC-351125, ancriviroc (formerly known as SCH-C), vicroviroc which has the chemical name (4,6-dimethylprymidine-5-yl){4-[(3S)-4-{(1R)-2-methoxy-1-[4-(trifluoromethyl)phenyl]ethyl}-3-methylpiperazin-1-yl]-4-methylpiperidin-1-yl}methanone, PRO-140, apliviroc (formerly known as GW-873140, Ono-4128, AK-602), AMD-887, INC-B9471, CMPD-167 which has the chemical name N-methyl-N-((1R,3S,4S)-3-[4- (3-benzyl-1-ethyl-1H-pyrazol-5-yl)piperidin-1-ylmethyl]-4-[3-fluorophenyl]cyclopent-1-yl]-D-valine), methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8- azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate and N-{(1S)-3-[3-endo-(5-Isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) and pharmaceutically acceptable salts, solvates or derivatives of the above. The last four compounds are disclosed in WO 03/084954 and WO 05/033107.

In a yet further embodiment, the CCR5 antagonist is selected from maraviroc, vicriviroc, NCB-9471, PRO-140, CCR5mAb004, 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, and N-{(1S)-3-[3-endo-(5-Isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) and pharmaceutically acceptable salts, solvates or derivatives of the above.

In a yet further embodiment, the CCR5 antagonist is maraviroc.

It is to be appreciated that the invention covers all combinations of embodiments as described herein.

Treatment experienced patients will be receiving an existing HIV therapy of one of more HIV drugs to control their viral load. Examples of HIV drug therapies include, but are not limited to, HIV protease inhibitors (PIs), non-nucleoside reverse transcriptase inhibitors (NNRTIs), nucleoside/nucleotide reverse transcriptase inhibitors (NRTIs), agents which inhibit the interaction of gp120 with CD4, other agents which inhibit the entry of HIV into a target cell (such as fusion inhibitors), inhibitors of HIV integrase, RNaseH inhibitors, prenylation inhibitors, maturation inhibitors which act by interfering with production of the HIV capsid protein.

It will be appreciated by a person skilled in the art, that a combination HIV therapy as referred to above may comprise two or more compounds having the same, or different, mechanism of action. Thus, by way of illustration only, a combination may comprise a CCR5 antagonist and one or more NRTIs; one or more NRTIs and a PI; one or more NRTIs and another CCR5 antagonist; a PI; a PI and an NNRTI; an NNRTI; and so on.

A recommended treatment for HIV is a combination drug treatment called Highly Active Anti-Retroviral Therapy, or HAART. HAART combines three or more HIV drugs.

In a yet further embodiment of the invention, the treatment experienced patient is receiving a HAART treatment regime comprising three or more HIV drugs, has a low viral load and a CCR5 antagonist is administered as a further agent to enhance the immune reconstitution of the patient.

Typically HAART therapy is selected from the following drug classes: HIV protease inhibitors (PIs), non-nucleoside reverse transcriptase inhibitors (NNRTIs), nucleoside/nucleotide reverse transcriptase inhibitors (NRTIs), and fusion inhibitors.

Examples of PIs include, but are not limited to, amprenavir (141W94), CGP-73547, CGP-61755, DMP-450 (mozenavir), nelfinavir, ritonavir, saquinavir, lopinavir, TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium, benzenesulfonamide derivatives disclosed in WO 03/053435, R-944, Ro-03-34649, VX-385, GS-224338, OPT-TL3, PL-100, PPL-100, SM-309515, AG-148, DG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005.

Examples of NRTIs include, but are not limited to, abacavir, GS-840, lamivudine, adefovir dipivoxil, beta-fluoro-ddA, zalcitabine, didanosine, stavudine, zidovudine, tenofovir disoproxil fumarate, amdoxovir (DAPD), SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine (FTC), Truvada (tenofovir and emtricitabine).

Examples of NNRTIs include, but are not limited to, efavirenz, HBY-097, nevirapine, TMC-120 (dapivirine), TMC-125, etravirine, delavirdine, DPC-083, DPC-961, capravirine, rilpivirine, TMC-278, Epzicom (abacavir and lamivudine), Trizivir (zidovudine and lamivudine and abacavir), Combivir (zidovudine and lamivudine), 5-{[3,5-Diethyl-1-(2-hydroxyethyl)-1H-pyrazol-4-yl]oxy}isophthalonitrile or pharmaceutically acceptable salts, solvates or derivatives thereof; GW-678248, GW-695634, MIV-150, calanolide, and tricyclic pyrimidinone derivatives as disclosed in WO 03/062238.

Examples of agents which inhibit gp120 and fusion inhibitors include, but are not limited to, BMS-806, BMS-488043, 5-{(1S)-2-[(2R)-4-Benzoyl-2-methyl-piperazin-1-yl]-1-methyl-2-oxo-ethoxy}-4-methoxy-pyridine-2-carboxylic acid methylamide and 4-{(1S)-2-[(2R)-4-Benzoyl-2-methyl-piperazin-1-yl]-1-methyl-2-oxo-ethoxy}-3-methoxy-N-methyl-benzamide, enfuvirtide (T-20), sifuvirtide, SP-01A, T1249, PRO 542, TNX-355, 2F5, 2G12, BMS-378806, BMS-488043, PRO-2000, DEBIO-025, PS-Ons, D5, TR-290999, TR-291144, AMD-3100, Soluble CD4, compounds disclosed in JP 2003171381, and compounds disclosed in JP 2003119137.

Examples of inhibitors of HIV integrase include, but are not limited to, L-000870810 GW-810781, 1,5-naphthyridine-3-carboxamide derivatives disclosed in WO 03/062204, compounds disclosed in WO 03/047564, compounds disclosed in WO 03/049690, and 5-hydroxypyrimidine-4-carboxamide derivatives disclosed in WO 03/035076, MK-0518, and GS-9137 (JTK-303), compounds disclosed in PCT/IB2006/002735, (5-(1,1-dioxo-1,2-thiazinan-2-yl)-N-(4-fluorobenzyl)-8-hydroxy-1,6-naphthyridine-7-carboxamide—disclosed in WO 03016315), GSK-364735.

Examples of prenylation inhibitors include, but are not limited to, HMG CoA reductase inhibitors, such as statins (e.g. atorvastatin).

Examples of maturation inhibitors include 3-O-(3′,3′-dimethylsuccinyl)betulic acid (otherwise known as PA-457) and alphaHGA.

Other therapeutic agents for use in combination with the CCR5 antagonist according to any aspect of the invention for enhanced treatment of HIV-defined opportunistic conditions include:

Anti-infectives (including antibacterials and antifungals). Examples of antibacterials include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R51211, trimetrexate, daunorubicin, recombinant human erythropoietin, recombinant human growth hormone, megestrol acetate, testerone, and total enteral nutrition. Examples of antifungals include, but are not limited to, anidulafungin, C31G, caspofungin, DB-289, fluconazaole, itraconazole, ketoconazole, micafungin, posaconazole, and voriconazole.

Agents useful in the treatment of hepatitis, such as interferons, pegylated interferons (e.g. peginterferon alfa-2a and peginterferon alfa-2b), long-acting interferons (e.g. albumin-interferon alfa), lamivudine, ribavirin, emtricitabine, viramidine, celgosivir, valopicitabine, HCV-086, HCV-796, EMZ702, BILN2061, IDN6566, NM283, SCH 6 and VX-950; serine inhibitors as disclosed in WO 05/007681; arylthiourea derivatives as disclosed in WO 05/007601, purine nucleoside analogues as disclosed in WO 05/009418, imidazole derivatives as disclosed in WO 05/012288, aza-peptide-based macrocyclic derivatives as disclosed in WO 05/010029.

Agents useful in the treatment of AIDS related Kaposi's sarcoma, such as interferons, daunorubicin, doxorubicin, paclitaxel, metallo-matrix proteases, A-007, bevacizumab, BMS-275291, halofuginone, interleukin-12, rituximab, porfimer sodium, rebimastat, COL-3.

Agents useful in the treatment of cytomegalovirus (CMV), such as fomivirsen, oxetanocin G, cidofovir, cytomegalovirus immune globin, foscarnet sodium, Isis 2922, valacyclovir, valganciclovir, ganciclovir.

Agents useful in the treatment of herpes simplex virus (HSV), such as acyclovir, penciclovir, famciclovir, ME-609.

There is also included within the scope the present invention, combinations of a CCR5 antagonist according to the invention together with one or more additional therapeutic agents independently selected from the group consisting of:

Proliferation inhibitors, e.g. hydroxyurea.

Immunomodulators, such as AD-439, AD-519, alpha interferon, AS-101, bropirimine, acemannan, CL246,738, EL10, FP-21399, gamma interferon, granulocyte macrophage colony stimulating factor (e.g. sargramostim), IL-2, immune globulin intravenous, IMREG-1, IMREG-2, imuthiol diethyl dithio carbamate, alpha-2 interferon, methionine-enkephalin, MTP-PE, remune, rCD4, recombinant soluble human CD4, interferon alfa-2, SK&F106528, soluble T4 thymopentin, tumor necrosis factor (TNF), tucaresol, recombinant human interferon beta, interferon alfa n-3.

Tachykinin receptor modulators (e.g. NK1 antagonists) and various forms of interferon or interferon derivatives.

Other chemokine receptor agonists/antagonists such as CXCR4 antagonists (e.g AMD070 and AMD3100) or CD4 antagonists (e.g. TNX-355).

Agents which substantially inhibit, disrupt or decrease viral transcription or RNA replication such as inhibitors of tat (transcriptional trans activator) or nef (negative regulatory factor).

Agents which substantially inhibit, disrupt or decrease translation of one or more proteins expressed by the virus (including, but not limited to, down regulation of protein expression or antagonism of one or more proteins) other than reverse transcriptase, such as Tat or Nef.

Agents which interfere with cell activation or cell cycling, such as rapamycin.

Other therapeutic agents may be used with the CCR5 antagonist in accordance with all aspects of the invention, e.g., in order to provide further immune stimulation or to treat pain and inflammation which accompany the initial and fundamental HIV infection.

Further combinations for use with a CCR5 antagonist according to all aspects of the invention include a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab).

In the above-described combinations, the CCR5 antagonist and additional therapeutic may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Pharmaceutically acceptable salts of the CCR5 antagonists listed herein include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002), incorporated herein by reference.

CCR5 antagonists may be administered alone or in combination with one or more other therapeutic compounds. Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of CCR5 antagonists and combinations thereof and methods for their preparation will be readily apparent to those skilled in the art. Such compositions and methods for their preparation may be found, for example, in Remington's Pharmaceutical Sciences, 19th Edition (Mack Publishing Company, 1995), incorporated herein by reference.

Suitable modes of administration include oral, parenteral, topical, inhaled/intranasal, rectal/intravaginal, and ocular/aural administration.

The CCR5 antagonists and combinations thereof may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The CCR5 antagonists and combinations thereof may also be used in fast-dissolving, fast-disintegrating dosage forms such as those described in Expert Opinion in Therapeutic Patents, 11 (6), 981-986, by Liang and Chen (2001), incorporated herein by reference.

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980), incorporated herein by reference.

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864, incorporated herein by reference. Details of other suitable release technologies such as high energy dispersions and osmotic and coated particles are to be found in Pharmaceutical Technology On-line, 25(2), 1-14, by Verma et al (2001), incorporated herein by reference. The use of chewing gum to achieve controlled release is described in WO 00/35298, incorporated herein by reference.

The CCR5 antagonists and combinations thereof may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

Formulations of CCR5 antagonsts also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999), incorporated herein by reference.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The CCR5 antagonists and combinations thereof can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of the invention. The overall daily dose will typically be in the range 1 μg to 200 mg which may be administered in a single dose or, more usually, as divided doses throughout the day.

The CCR5 antagonists and combinations may be administered rectally or vaginally, for example, in the form of a suppository, pessary, vaginal ring, microbicide or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The CCR5 antagonists and combinations thereof may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

CCR5 antagonists and combinations thereof may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148, incorporated herein by reference.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

In a further aspect of the invention, there is provided a method of treating HIV in a patient infected with a CXCR4 using viral population, comprising administering a beneficial amount of a CCR5 antagonist, wherein the viral population of said patient contains more than 2% CXCR4 virus. In further embodiments, the viral population of the patient contains more than 5%, 10%, 15%, 20%, 25%, 30%, 35%, 40%, 45% or 50% of CXCR4 virus. The patient can be treatment naive or treatment experienced. In a further embodiment, the CCR5 antagonist is maraviroc.

In a yet further aspect of the invention, there is provided a method of increasing the CD4, or CD8, or both CD4 and CD8 count in an HIV patient infected with a CXCR4 using viral population comprising administering a CCR5 antagonist.

In a yet further aspect of the invention, there is provided a method of increasing the CD4 count in an HIV patient infected with a CXCR4 using viral population comprising administering a beneficial amount of a CCR5 antagonist, wherein the viral population of said patient contains more than 2% CXCR4 virus. In further embodiments, the viral population of the patient contains more than 5%, 10%, 15%, 20%, 25%, 30%, 35%, 40%, 45% or 50% of CXCR4 virus. The patient can be treatment naive or treatment experienced. In a further embodiment, the CCR5 antagonist is maraviroc.

In a yet further aspect of the invention, there is provided a method of increasing the CD8 count in a an HIV patient infected with a CXCR4 using viral population comprising administering a beneficial amount of a CCR5 antagonist, wherein the viral population of said patient contains more than 2% CXCR4 virus. In further embodiments, the viral population of the patient contains more than 5%, 10%, 15%, 20%, 25%, 30%, 35%, 40%, 45% or 50% of CXCR4 virus. The patient can be treatment naive or treatment experienced. In a further embodiment, the CCR5 antagonist is maraviroc.

In a yet further aspect of the invention, there is provided a method of increasing the CD4 and CD8 count in an HIV patient infected with a CXCR4 using viral population comprising administering a CCR5 antagonist, wherein the viral population of said patient contains more than 2% CXCR4 virus. In further embodiments, the viral population of the patient contains more than 5%, 10%, 15%, 20%, 25%, 30%, 35%, 40%, 45% or 50% of CXCR4 virus. The patient can be treatment naive or treatment experienced. In a further embodiment, the CCR5 antagonist is maraviroc.

In a yet further aspect of the invention, there is provided a method of enhancing immune reconstitution in an HIV patient infected with a CXCR4 using viral population comprising administering to the patient a beneficial amount of a CCR5 antagonist. In further embodiments the viral population of the patient contains more than 2%, 5%, 10%, 15%, 20%, 25%, 30%, 35%, 40%, 45% or 50% of CXCR4 virus. The patient can be treatment naive or treatment experienced. In a further embodiment, the CCR5 antagonist is maraviroc.

In a yet further aspect of the invention, there is provided a method of treating an HIV related opportunistic infection in an HIV patient infected with a CXCR4 using viral population comprising administering to the patient a beneficial amount of a CCR5 antagonist. In further embodiments the viral population of the patient contains more than 2%, 5%, 10%, 15%, 20%, 25%, 30%, 35%, 40%, 45% or 50% of CXCR4 virus. The patient can be treatment naive or treatment experienced. In a further embodiment, the CCR5 antagonist is maraviroc.

In a further aspect of the invention, there is provided use of a CCR5 antagonist for increasing the CD4, or CD8, or both CD4 and CD8 cell count in a patient infected with a CXCR4 using viral population, wherein the viral population of said patient contains more than 2% CXCR4 virus.

In a further aspect of the invention, there is provided use of a CCR5 antagonist enhance immune reconstitution in a patient infected with a CXCR4 using viral population, wherein the viral population of said patient contains more than 2% CXCR4 virus.

It will be appreciated that in all the above asects and embodiments of the invention where “use of a CCR5 antagonist in the preparation of a medicament” are defined, that corresponding aspects and embodiments of “a CCR5 antagonist for use in . . . ” are also within the scope of the invention.

For example, in a further aspect of the invention, there is provided a CCR5 antagonist for use in increasing the CD4, or CD8, or both CD4 and CD8, cell count of an HIV patient infected with a CXCR4 using viral population. In further embodiments the viral population of the patient contains more than 2%, 5%, 10%, 15%, 20%, 25%, 30%, 35%, 40%, 45% or 50% of CXCR4 virus. The patient can be treatment naive or treatment experienced. In a further embodiment, the CCR5 antagonist is maraviroc.

A phenotypic assay for HIV-1 is described in Antimicrobial Agents and Chemotherapy, April 2000, p 920-928.

The invention will now be described by way of example by reference to the accompanying example.

### EXAMPLE

Treatment experienced HIV-1 patients infected with a CXCR4 using viral population were selected according to the following protocol and the first group with optimised background therapy (OBT) alone was compared against groups on OBT plus maraviroc once a day and OBT plus maraviroc twice a day.

### Selection Criteria

### Patients Enrolled in the Trial:

(a) Were aged 16 or over;

(b) were infected with a non R5 tropic (CXCR4 using viral population) as determined by Monogram Bioscience Phenosense™ assay (WO 02/-99383; U.S. Pat. No. 5,837,464), or were of indeterminate tropism phenotype;

(c) had been on a stable antiviral regimen for at least 4 weeks prior to randomisation:

(d) had an HIV-1 RNA count of at least 5,000 copies/mL as measured by the Roche Amplicor HIV-1 Monitor (version 1.5)

(e) (i) had at least three months previous antiretroviral experience with at least one agent from three of the four antiretroviral drug classes: NTRIs, NNRTIs, protease inhibitors and fusion inhibitors (i.e. were triple class experienced); or

(ii) had documented resistance to members of two of the four antiretroviral drug classes (i.e. were dual class resistant).

### Trial Treatments

The eligible patients were randomised into three groups based on the drug regimens they received.

Group 1: dptimised background therapy (OBT) (3-6 antiretroviral drugs [not counting low dose retonavir] of which at least one is active and no more than one is an NNRTI) plus maraviroc 150 mg po taken once daily (QD).

Group 2: optimised background therapy (as above) plus maraviroc 150 mg po taken twice daily (BID).

Group 3: optimised background therapy (as above) plus placebo.

Patients whose optimised background therapy did not contain a protease inhibitor (PI) or delavirdine (an NNRTI) were randomised to receive 300 mg doses of maraviroc once or twice daily.

Patients were stratified according to whether they had an HIV-1 RNA count of greater than or less than 100,000 copies/mL and were receiving enfuvirtide as part of their optimised background therapy. These patients were distributed evenly among the 3 patient groups.

Several measurements of viral load (VL) and CD4 count were taken for each patient before the start of any treatment with maraviroc or placebo. The mean of these measurements was taken to be the baseline measurement for each patient. The mean of the baseline measurement of all patients for viral load and CD4 count are shown in table 1. The median viral load and CD4 count for all patients (before start of treatment) are also shown in table 1.

After 24 weeks of treatment, the endpoints (as discussed below) were measured.

(a) For each patient, the change from baseline in HIV-1 viral load (on a log[base10]scale) was measured. The mean of these values (the mean change in viral load for all patients) is shown in table 2.

(b) The percentage of patients with fewer than (i) 400 and (ii) 50 HIV-1 RNA copies\mL.

(c) The percentage of patients who had a viral load reduction from baseline of at least 0.5 or 1.0log₁₀ copies/mL.

(d) For each patient, the change from baseline in CD4 and CD8 cell count was measured. The mean of these values is shown in table 2.

Cell count was measured by fluorescence activated cell sorting (FACS), which is a standard technique.

TABLE 1
Mean and Median CD4 Cell Count and HIV-1 RNA level

| | Placebo + | Once Daily maraviroc + | Twice Daily maraviroc + |
|---|---|---|---|
| Variable | Optimised Regimen | Optimised Regimen | Optimised Regimen |
| CD4 Mean (cells\μL) | 98.6 | 85.0 | 96.4 |
| CD4 Median (cells\μL) | 41.5 | 39.5 | 43.1 |
| Mean VL (log₁₀ | 5.01 | 5.03 | 5.10 |
| copies/mL) | | | |
| Median VL (log₁₀ | 5.10 | 5.01 | 5.17 |
| copies/mL) | | | |

As can be seen from table 1, all patient groups started with similar CD4 cell numbers

TABLE 2
Measurements after 24 weeks

| | Placebo + | Once Daily maraviroc + | Twice Daily maraviroc + |
|---|---|---|---|
| Variable | Optimised Regimen | Optimised Regimen | Optimised Regimen |
| Mean VL reduction | −0.97 | −0.91 | −1.20 |
| (log₁₀ copies/mL) | | | |
| Treatment difference in | | +0.06 | −0.23 |
| VL reduction (log₁₀ | | (−0.53, +0.64) | (−0.83, +0.36) |
| copies/mL) 97.5% | | | |
| confidence interval | | | |
| VL < 400 counts/mL | 24.1% | 24.6% | 30.8% |
| VL < 50 counts\mL | 15.5% | 21.0%\hat{ } | 26.9% |
| >0.5 log₁₀ VL reduction | 39.7% | 42.1% | 48.1% |
| >1 log₁₀ VL reduction | 36.2% | 31.6% | 44.2% |
| Mean change in CD4 | +35.7 | +59.6 | +62.4 |
| count | | | |
| Mean change in CD8 | +150.0 | +384.5 | +338.8 |
| count | | | |

### RESULTS

As can be seen from the pre-treatment measurements in table 1, median CD4 count was less than 45 cells/μL and mean baseline viral load was greater than 5[log10]counts/mL for each treatment group. As shown in table 2, viral load change from baseline was similar for the maraviroc once daily and twice daily groups (−1.2 log₁₀) and placebo groups (−0.97 log₁₀). Adverse clinical and laboratory events, discontinuations due to adverse events and deaths occurred with similar frequency in all three groups. There were no cases of lymphoma or adenocarcinoma.

There is a clinically meaningful (and statistically significant) increase in CD4 and CD8 cell count in those patients receiving maraviroc both once and twice daily as compared to the placebo. As shown in table 1, mean baseline CD4 counts for all three treatment groups were less than 100 mL and slightly, but not statistically lower, in the once daily maraviroc group (85 cells/μL) compared to the twice daily maraviroc group (96.4 cells/μL) and the placebo group (98.6 cells/μL). As shown in table 2, mean CD4 change was greater for maraviroc once and twice daily groups: +59.6 cells/μL (once daily) and +62.2 cells/μL (twice daily), compared to +35.4 cells/μL for placebo. Mean CD8 cell change was also greater for the maraviroc groups +384.5 cells/μL (once daily) and +338.8 cells/μL (twice daily) compared to placebo (+150.0 cells/μL).Thus there is a clinically meaningful numerical difference in the size of increase of both maraviroc groups as compared to placebo group. This shows that maraviroc can be given to HIV patients infected with a CXCR4 using viral population to induce a clinically meaningful increase in their CD4, or CD8, or both CD4 and CD8, cell count over optimised background treatment alone. This indicates that maraviroc enhances the immune reconstitution of the HIV patients and so can be used for the treatment or prophylaxis of HIV related opportunistic conditions.

1. (canceled)

2. (canceled)

3. (canceled)

4. (canceled)

5. The method according to claim 35, wherein the HIV related opportunistic condition is selected from pneumocystitis carnii, toxoplasmosis, isoporiasis, cryptosporidiosis, cadidiasis, cryptococcosis, histoplasmosis, coccidioidomycosis, mycobacterium tuberculosis, non tuberculosis mycobacterium infections, salmonella, cytomegalovirus, herpes simplex virus, progressive multifocal leukoencephalopathy, Kaposi's sarcoma, lymphoma, cervical carcinoma, HIV dementia, HIV wasting syndrome, recurrent or persistent upper respiratory infection, sinuisitis, otitis media, bacterial meningitis, pneumonia, sepsis, oropharyngis candidaiasis, diarrhea, hepatitis, herpes zoster, leiomyosarcoma, lymphoid interstiticial pneumonia, nocardiosis, disseminated varicella, and toxoplasmosis of the brain.

6. The method according to claim 35, wherein before administration of the CCR5 antagonist the patient has a baseline CD4 count of less than 200 cells/μL.

7. (canceled)

8. The method according to claim 35 wherein the CD4 count of the patient after treatment with the CCR5 antagonist is more than 50 cells/μL.

9. (canceled)

10. (canceled)

11. (canceled)

12. The method according to claim 35 wherein the CD4 count of the patient is increased by more than 60% after treatment with the CCR5 antagonist.

13. (canceled)

14. (canceled)

15. The method according to claim 35 wherein the patient is treatment experienced.

16. The method according to claim 15 wherein the treatment experienced patient has a viral load of less than 5000 copies/mL.

17. (canceled)

18. (canceled)

19. The method according to claim 15 wherein the treatment experienced HIV patient is receiving an existing HMART treatment regime comprising three or more HIV drugs.

20. The method according to claim 35 wherein the patient is infected with a CXCR4 using viral population.

21. The method according to claim 20 wherein the viral population of the patient contains more than 2% CXCR4 virus.

22. (canceled)

23. (canceled)

24. (canceled)

25. (canceled)

26. The method according to claim 35 wherein the patient is infected with a CCR5 tropic viral population.

27. The method according to claim 35 wherein the patient is infected with a CCR5 tropic viral population and wherein the patient is treatment experienced and has a low viral load and low CD4 cell count and the CCR5 antagonist is given in addition to their existing HIV therapy to increase their CD4 cell count.

28. The method according to claim 27 wherein the patient has a viral load of less than 400 copies/mL and a CD4 cell count of less than 200 cells/μL.

29. The method according to claim 35 wherein the CCR5 anatagonist is selected from maraviroc, vicriviroc, NCB-9471, PRO-140, 8-[4-(2-butoxyethoxy)phenyl]-1-isobutyl-N-[4-[[(1-propyl-1H-imadazol-5-yl)methyl]sulphinyl]phenyl]- 1,2,3,4-tetrahydro-1-benzacecine-5-carboxamide, methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H -imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5carboxylate, and N-{(1S)3-[3-endo-(5-Isobutyryl-2-methyl4,5,6,7-tetrahydro-1H-imidazo[4,5-c]pyridin-1-yl)-8-azabicyclo[3.2.1]oct-8-yl]-1-(3-fluorophenyl)propyl}acetamide); and pharmaceutically acceptable salts, solvates or derivatives thereof.

30. The method according to claim 35 wherein the CCR5 antagonist is maraviroc or a pharmaceutically acceptable salt or solvate thereof.

31. The method according to claim 35 wherein the CCR5 antagonist is indicated to be administered without requiring that the viral tropism of the patient is previously determined.

32. (canceled)

33. A method for enhancing immune reconstitution in an HIV patient comprising administering to the patient a beneficial amount of a CCR5 antagonist.

34. A method of increasing the CD4, or CD8, or both CD4 and CD8, cell count of an HIV patient comprising administering to the patient a beneficial amount of a CCR5 antagonist.

35. A method for the treatment of an HIV related opportunistic condition in an HIV patient comprising administering to the patient a beneficial amount of a CCR5 antagonist.
